(19) United States
(12) Patent Application Publication
(10) Pub. No.: US 2017/0258476 A1
(43) Pub. Date: Sep. 14, 2017

(54) BLOOD VESSEL TREATMENT METHOD

(71) Applicant: TERUMO KABUSHIKI KAISHA, Tokyo (JP)

(72) Inventors: Kouichi HAYAKAWA, Tokyo (JP); Yuri AKIMOTO, Hadano-city (JP)

(73) Assignee: TERUMO KABUSHIKI KAISHA, Tokyo (JP)

(21) Appl. No.: 15/064,094

(22) Filed: Mar. 8, 2016

(51) Int. Cl. A61B 17/12; A61M 25/00

(52) U.S. Cl. CPC A61B 17/12186; A61B 17/12109; A61M 25/007; A61B 2017/1205

(57) ABSTRACT

A blood vessel treatment method including inserting a medical device into a blood vessel, the medical device including an elongate shaft portion and a contact portion configured to contact a biological tissue in the blood vessel; bringing the contact portion into contact with the biological tissue in the blood vessel; twisting the blood vessel by moving the contact portion; and releasing a treatment agent from the medical device for occluding or contracting a lumen of the blood vessel.

### CROSS-REFERENCES TO RELATED APPLICATIONS

This application is based on and claims priority to Japanese Application No. JP 2015/049436 filed on Mar. 12, 2015, the entire content of which is incorporated herein by reference.

### TECHNICAL FIELD

The present disclosure relates to a blood vessel treatment method for treating a blood vessel. More particularly, the disclosure relates to a blood vessel treatment method for occluding or contracting a varicose vein.

### BACKGROUND DISCUSSION

Veins are provided with venous valves for preventing the backflow of blood. Venous valves in a lower limb of a living body are contracted by muscles of the lower limb, to function also as a pump for returning blood to the heart against the force of gravity.

When a venous valve becomes unable to operate normally, backflow of blood occurs in the vein and the vein enlarges, which may cause a varicose vein. Varicose veins often occur in the great saphenous vein and the small saphenous vein. The great saphenous vein and the small saphenous vein are superficial veins (i.e., closer to the surface of the living body than deep veins) in a lower limb that are subject to high pressures when the living body is in a standing position. When a venous valve in the superficial vein is operating normally, blood flows from the superficial vein into a deep vein. If the venous valve in the superficial vein stops operating normally, blood flows reversely from the deep vein into the superficial vein, whereby the superficial vein is enlarged, causing a tortuous varicose vein.

Treating methods for varicose veins in a lower limb include removing the vein itself (stripping) and occluding the vein. Examples of methods for occluding the vein include externally compressing the vein, a high ligation method to ligate the vein at a high position to prevent backflow of blood, an ablation method to occlude the vein by thermal cauterization using RF (radiofrequency wave) or a laser, using a treatment agent such as a sclerosing agent, an adhesive, etc. to occlude the varicose vein, and stimulating the inner wall of the varicose vein to occlude the blood vessel.

In the therapy conducted using a treatment agent, damage to a blood vessel or inflammation of the blood vessel is induced by the treatment agent to cause thrombus formation and adhesion of the blood vessel inner wall, thereby occluding the vein, interrupting the blood flow and/or degenerating the vein.

### SUMMARY

A liquid treatment agent is influenced by the flow of blood and, hence, does not tend to stay at a part to be treated (i.e., the blood flow carries the liquid treatment agent away from the part to be treated). In view of this, a treatment agent may be injected into a vein after the blood flow in the vein is temporarily interrupted or reduced by compressing the vein with a balloon or from outside the living body (e.g., pressure is applied on the limb at a position overlying the great saphenous vein). Alternatively, a treatment agent may be foamed to lower its fluidity and then injected into the blood vessel to permit the treatment agent to remain at the part to be treated for a longer time. If the blood flow is intercepted or reduced, or the treatment agent is foamed, however, the presence of blood in the blood vessel may cause the treatment agent to be diffused by the blood, lowering the effect of the treatment agent.

The blood vessel treatment method disclosed here permits a treatment agent to effectively occlude or contract a blood vessel.

In one aspect of the disclosure here, there is provided a method for inserting a medical device into a blood vessel, the medical device including an elongate shaft portion and a contact portion configured to contact a biological tissue in the blood vessel; bringing the contact portion into contact with the biological tissue in the blood vessel; twisting the blood vessel by moving the contact portion; and releasing from the medical device a treatment agent for occluding or contracting a lumen of the blood vessel. Another aspect of the disclosure here involves a method including inserting a medical device into a blood vessel of a living body; twisting the blood vessel so that the inner diameter of the blood vessel narrows where the medical device is located; and releasing a treatment agent from the medical device while the blood vessel is twisted and the inner diameter of the blood vessel is narrowed where the medical device is located. Another aspect of the disclosure here involves a method including gripping a blood vessel of a living body, the blood vessel possessing an inner diameter; twisting the blood vessel so that the inner diameter of the blood vessel narrows at a twisting location; and releasing a treatment agent at the twisting location.

In the blood vessel treatment methods described above, the treatment agent can be released in a condition where the blood flow in the blood vessel is interrupted or reduced and the amount of blood in the blood vessel is reduced by the twisting of the blood vessel. Therefore, diffusion of the treatment agent can be restrained, and the treatment agent can be made to act effectively on the blood vessel wall. Consequently, the blood vessel can be effectively occluded or contracted.

### BRIEF DESCRIPTION OF THE DRAWINGS

FIGS. 1A and 1B are plan views of a medical device according to a first embodiment, wherein FIG. 1A depicts an initial state before expansion of the contact portion, and FIG. 1B depicts a state after expansion of the contact portion;

FIGS. 2A and 2B are sectional views of the medical device of the first embodiment, wherein FIG. 2A depicts an initial state before expansion of the contact portion, and FIG. 2B depicts a state after expansion of the contact portion;

FIGS. 3A and 3B are sectional views illustrating the medical device of the first embodiment inserted in a vein, wherein FIG. 3A depicts an initial state before expansion of the contact portion, and FIG. 3B depicts a state after expansion of the contact portion;

FIGS. 4A and 4B are sectional views illustrating the medical device of the first embodiment is inserted in a vein, wherein FIG. 4A depicts a state where the vein is twisted, and FIG. 4B depicts a state when fluid is released and a blood vessel wall is damaged by the contact portion;

FIG. 5 is a sectional view taken along line A-A of FIG. 4A;

FIGS. 6A and 6B are plan views illustrating a modification of the medical device of the first embodiment, wherein FIG. 6A depicts an initial state before expansion of the contact portion, and FIG. 6B depicts a state after expansion of the contact portion;

FIGS. 7A and 7B are plan views illustrating a second embodiment of the medical device, wherein FIG. 7A depicts an initial state before expansion of the contact portion, and FIG. 7B depicts a state after expansion of the contact portion;

FIGS. 8A and 8B are sectional views of the second embodiment of the medical device, wherein FIG. 8A depicts an initial state before expansion of the contact portion, and FIG. 8B depicts a state after expansion of the contact portion;

FIGS. 9A and 9B are sectional views illustrating the second embodiment of the medical device inserted in a vein, wherein FIG. 9A depicts a state before expansion of a contact portion, and FIG. 9B depicts a state after expansion of the contact portion;

FIGS. 10A and 10B are sectional views illustrating the second embodiment of the medical device inserted in a vein, wherein FIG. 10A depicts a state where of the vein is twisted, and FIG. 10B depicts a state when fluid is released and a blood vessel wall is damaged by the contact portion;

FIGS. 11A and 11B illustrate a state after expansion of a contact portion of a third embodiment of the medical device, wherein FIG. 11A is a plan view, and FIG. 11B is a sectional view;

FIGS. 12A and 12B are sectional views illustrating a third embodiment of the medical device inserted in a vein, wherein FIG. 12A depicts an initial state before expansion of a contact portion, and FIG. 12B depicts a state after expansion of the contact portion;

FIGS. 13A and 13B are sectional views illustrating the third embodiment of the medical device inserted in a vein, wherein FIG. 13A depicts a state where the vein is twisted, and FIG. 13B depicts a state when fluid is released;

FIG. 14 is a sectional view illustrating another method of using the third embodiment of the medical device;

FIGS. 15A and 15B are sectional views illustrating a first modification of the third embodiment of the medical device inserted in a vein, wherein FIG. 15A depicts an initial state before expansion of a contact portion, and FIG. 15B depicts a state after expansion of the contact portion;

FIGS. 16A and 16B are sectional views illustrating a second modification of the third embodiment of the medical device inserted in a vein, wherein FIG. 16A depicts an initial state before expansion of a contact portion, and FIG. 16B depicts a state after expansion of the contact portion; and

FIG. 17 is a sectional view illustrating a third modification of the third embodiment of the medical device.

### DETAILED DESCRIPTION

Set forth below is a detailed description of embodiments of a medical device for treating a blood vessel and a method for treating a blood vessel representing examples of the inventive medical device and method disclosed here. Note that the dimensional ratios in the drawings may be exaggerated for convenience of explanation and may therefore be different from the actual ratios.

A medical device 1 according to a first embodiment disclosed herein is used for treatment of veins (i.e., a type of blood vessel), such as for occluding or contracting a varicose vein. Varicose veins occur mainly in lower limb veins, particularly in superficial veins such as great saphenous veins and small saphenous veins. The varicose veins can also occur in pelvic, ovarian and spermatic cord veins. The medical device 1 is used to stimulate the inner wall of a vein to damage the vein inner wall. The vein inner wall may be further damaged by injecting a treatment agent, such as a sclerosant or an adhesive, into the vein to occlude or contract the vein, thereby inhibiting backflow of blood. Note that the side toward which the device is inserted into a vein is referred to here as the “distal side” or “distal end” and the side of an operator's proximal operation is referred to as the “proximal side” or “proximal end.” The “initial state” as used below is the state before expansion of a contact portion 30.

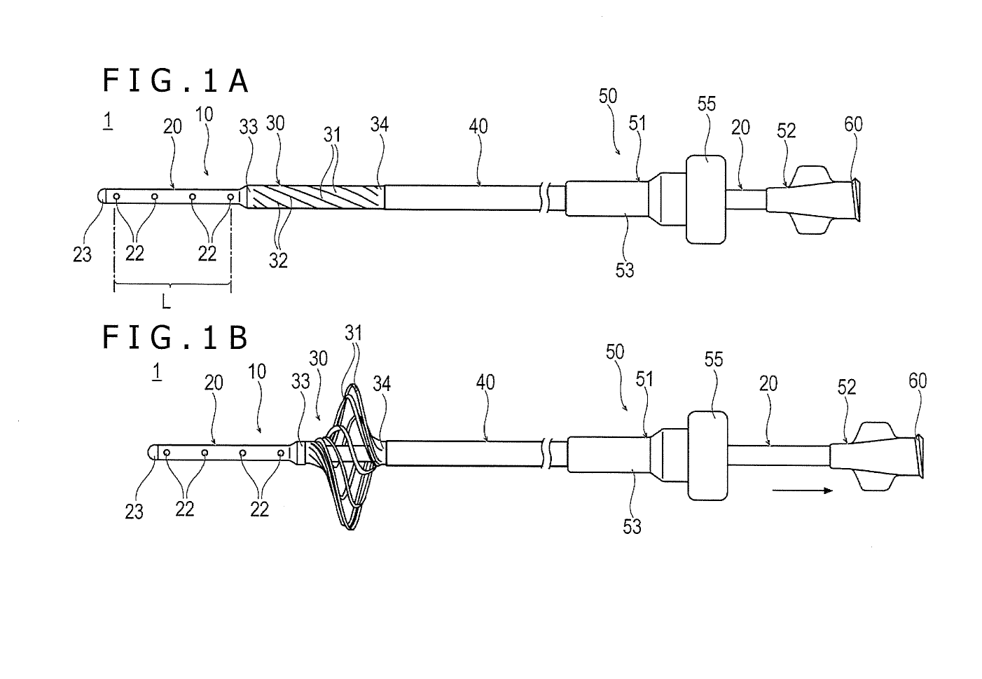

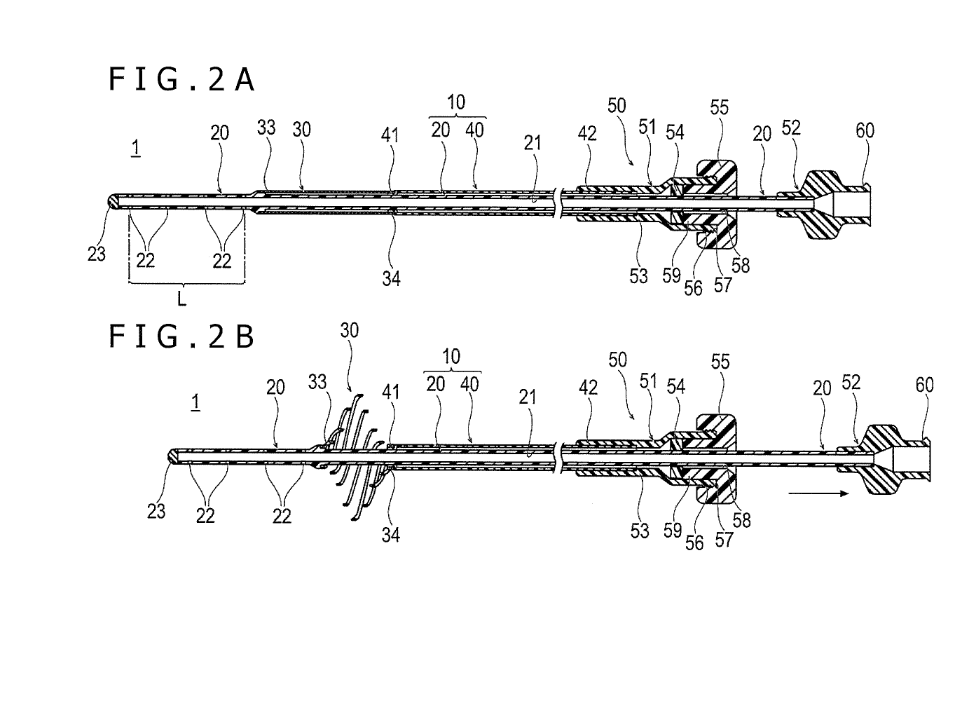

The medical device 1 depicted in FIGS. 1A to 2B includes an elongate shaft portion 10, a contact portion 30 capable of expansion and contraction at a distal portion of the shaft portion 10, and an operating portion 50 for expanding and contracting the contact portion 30. The shaft portion 10 includes a pipe shaped (i.e., cylindrically shaped) inner tube 20, and a pipe shaped outer sheath 40 accommodating the inner tube 20.

The inner tube 20 includes a first lumen 21 for passage therethrough of a treatment agent such as a sclerosant or an adhesive (i.e., the treatment agent flows within the first lumen 21). Examples of the treatment agent include polidocanol, sodium tetradecyl sulfate (STS), and cyanoacrylate. The inner tube 20 is on the distal side of the contact portion 30 with inner tube side holes 22 piercing from the inner surface to the outer surface of the inner tube 20 (i.e., the inner tube side holes 22 pierce through the wall of the inner tube 20). A tip member 23 is secured to a distal portion of the inner tube 20 for closing the inner tube 20 (i.e., to prevent fluids that are external to the inner tube 20 from entering the inner tube 20). A proximal portion of the inner tube 20 is fixed to a second operating portion 52 of the operating portion 50. The operating portion 50 includes a first operating portion 51 and the second operating portion 52. The first operating portion 51 is located proximally of the second operating portion 52.

The outer sheath 40 is a pipe body accommodating the inner tube 20 and is movable in an axial direction relative to the inner tube 20. The distal end portion 41 of the outer sheath 40 is slidable in close contact with the outer peripheral surface of the inner tube 20, and the proximal end portion 42 of the outer sheath 40 is fixed to the first operating portion 51 of the operating portion 50.

The distal end portion 33 of the contact portion 30 is secured to a distal portion of the inner tube 20, and the proximal end portion 34 of the contact portion 30 is secured to the distal end portion 41 of the outer sheath 40. The contact portion 30 includes a plurality of spirally shaped linear material portions 31 aligned in the circumferential direction of the contact portion 30. Each of the linear material portions 31 can bend outward to expand and come away from the outer peripheral surface of the inner tube 20 (see FIGS. 1B and 2B). The linear material portions 31 expanded/bent outward by proximally moving the inner tube 20 relative to the outer sheath 40. From the expanded state, each linear material portion 31 can be contracted to come closer to the outer peripheral surface of the inner tube 20 (see FIGS. 1A and 2A) by distally moving the inner tube 20 relative to the outer sheath 40. The linear material portions 31 are formed from a pipe body with a plurality of spiral slits 32 (e.g., spiral slits 32 may be cut into a pipe body to form the linear material portions 31). Note that the linear material portions 31 of the contact portion 30 may also be formed by arraying wires.

The materials constituting the inner tube 20 and the outer sheath 40 are preferably hard, flexible materials. Examples include polyolefins such as polyethylene, polypropylene, etc., polyamides, polyesters such as polyethylene terephthalate, etc., fluoropolymers such as tetrafluoroethylene-ethylene (ETFE), etc., polyether-ether ketone (PEEK), polyimides, shape memory alloys provided with a shape memory effect or superelasticity by heat treatment, stainless steel, tantalum (Ta), titanium (Ti), platinum (Pt), gold (Au), tungsten (W), and so on. Preferable examples of the shape memory alloys include Ni—Ti alloys, Cu—Al—Ni alloys, and Cu—Zn—Al alloys. Metallic braids or coils may be added to the above-mentioned materials for the purpose of enhancing rigidity.

The material constituting the contact portion 30 is preferably a hard, flexible material. Examples include polyolefins such as polyethylene, polypropylene, etc., polyamides, polyesters such as polyethylene terephthalate, etc., fluoropolymers such as ETFE, etc., PEEK, polyimides, shape memory alloys provided with a shape memory effect or superelasticity by heat treatment, stainless steel, Ta, Ti, Pt, Au, W, and so on. Preferable examples of the shape memory alloys include Ni—Ti alloys, Cu—Al—Ni alloys, and Cu—Zn—Al alloys.

The length of the medical device 1 (the length from the distal portion of the inner tube 20 to the proximal end of the operating portion 50) is not particularly limited, but is preferably 100 mm to 1,000 mm, for example. The outside diameter of the outer sheath 40 is not specifically restricted, but is preferably 1.0 mm to 3.0 mm, for example. The inside diameter of the inner tube 20 is not particularly limited, but is preferably 0.3 mm to 1.0 mm, for example. The maximum outside diameter of the contact portion 30 in its expanded state is not specifically restricted, but is preferably 3.0 mm to 20 mm, for example. The length of the range L over which the inner tube side holes 22 of the inner tube 20 are formed is not particularly limited, but is preferably 30 mm to 200 mm, for example.

The contact portion 30, the inner tube 20 and/or the outer sheath 40 may be formed to contain a radiopaque material in the materials thereof. A radiopaque marker enables an operator to more accurately grasp the position of the medical device 1, and hence a user may have an easier procedure using radioscopy. Preferable examples of the radiopaque material include gold, platinum, platinum-iridium alloy, silver, stainless steel, molybdenum, tungsten, tantalum, palladium, and alloys of these materials.

In addition, the contact portion 30, the inner tube 20 and/or the outer sheath 40 may be formed to allow ultrasonic visual recognition. This enables an operator to more accurately grasp the position of the medical device 1, and hence a user may have an easier procedure when using an ultrasonic diagnosis apparatus. Examples of a structure to allow ultrasonic visual recognition include formation of ruggedness such as projections, recesses, minute grooves, or holes in the surfaces of the contact portion 30, the inner tube 20 and the outer sheath 40 so that the surface(s) is a rugged surface. Ultrasonic visual recognition also may be improved by adding, for example, porous material such as foam metal, cellular plastic, etc. to the outer surface of the contact portion 30, the inner tube 20, and/or the outer sheath 40.

A marker composed of a radiopaque material may be disposed at either one or both of the inner tube 20 and the outer sheath 40. For example, the radiopaque marker may be at a portion of the inner tube 20 surrounded by the contact portion 30. The marker may be attached, for example, by winding a wire formed from a radiopaque material around the outer surface of the inner tube 20. The marker may also be a pipe formed from a radiopaque material that is attached to the inner tube 20 by caulking or adhesion.

The operating portion 50 includes the first operating portion 51 connected to the proximal end portion 42 of the outer sheath 40, and the second operating portion 52 connected to the proximal end portion of the inner tube 20. The first operating portion 51 includes: an operating portion main body 53 fitted and connected to the proximal end portion 42 of the outer sheath 40; a seal portion 54 inside the operating portion main body 53; and a seal adjusting portion 55 connected to a proximal end portion of the operating portion main body 53 (i.e. a proximal end portion of the operating portion main body 53).

The operating portion main body 53 is a pipe shaped (i.e., cylindrically shaped) member. The proximal end portion of the inner tube 20 is fitted and connected to the inside of the distal end portion of the operating portion main body 53. The seal portion 54 is disposed on the inside of the distal end portion of the operating portion main body 53. The proximal end portion of the operating portion main body 53 is formed, at its outer peripheral surface, with a male screw portion 56 for screw engagement with the seal adjusting portion 55.

The seal portion 54 is an annular member capable of elastic deformation (i.e., the seal portion 54 may expand and compress). The inner tube 20 is inserted in and passed through the seal portion 54 (i.e., the seal portion 54 surrounds the outer diameter of the inner tube 20). The seal portion 54 permits insertion and passage of the inner tube 20 within the inner diameter of the seal portion 54, while maintaining liquid-tightness of the inside of the operating portion main body 53 (i.e., the inner tube 20 is slidable within the inner diameter of the seal portion 54).

The seal adjusting portion 55 is a pipe shaped member with a penetration hole 58. The inner tube 20 extends through the penetration hole 58 of the seal adjusting portion 55. The seal adjusting portion 55 includes a female screw portion 57 for screw engagement with the male screw portion 56 of the operating portion main body 53 and a pressing portion 59. The pressing portion 59 projects distally on the inside of the female screw portion 57 and is capable of (i.e., configured to) press the seal portion 54 (i.e., the pressing portion 59 is configured to contact and compress the seal portion 54). When the female screw portion 57 engages with the male screw portion 56 of the operating portion main body 53 and the seal adjusting portion 55 rotates, the pressing portion 59 presses against the seal portion 54 inside the operating portion main body 53, thereby contracting the inside diameter of the seal portion 54. The contraction of the inner diameter of the seal portion 54 causes the seal portion 54 to come into close contact with the outer peripheral surface of the inner tube 20 penetrating the seal portion 54, whereby liquid-tightness of the inside of the operating portion main body 53 can be maintained. By rotating the seal adjusting portion 55, the seal portion 54 can be brought into close contact with the outer peripheral surface of the inner tube 20 to fix the position of the inner tube 20 (i.e., the inner tube 20 will not move proximally or distally relative to the seal portion 54).

The second operating portion 52 is a pipe shaped member. The proximal end portion of the inner tube 20 is fitted and connected to the inside of a distal portion of the second operating portion 52. The proximal portion of the second operating portion 52 has an injection port 60 to which a three-way cock or a syringe or the like can be connected.

When the second operating portion 52 is moved proximally relative to the first operating portion 51, the distal end portion 33 and the proximal end portion 34 of the contact portion 30 move closer to one another, and the linear material portions 31 bend outward and expand away from the outer peripheral surface of the inner tube 20 (see FIGS. 1B and 2B). When the second operating portion 52 is moved distally relative to the first operating portion 51, the distal end portion 33 and the proximal end portion 34 of the contact portion 30 move away from one another, and the linear material portions 31 contract to move closer to the outer peripheral surface of the inner tube 20 (see FIGS. 1A and 2A).

The materials constituting the operating portion main body 53, the seal adjusting portion 55 and the second operating portion 52 are not particularly limited. Examples of applicable materials include rigid resins such as polycarbonate, polyethylene, polypropylene, etc.

The material constituting the seal portion 54 is not specifically restricted. Examples include natural rubber, silicone rubber, nitrile rubber, and fluororubber.

A method of using the medical device 1 according to the first embodiment will now be described, in reference to an example of occluding a vein V in the state of varicose vein occurring in a great saphenous vein or a small saphenous vein of a lower limb.

First, the medical device 1 is primed by flushing the inside of the inner tube 20, the contact portion 30, the outer sheath 40, the first operating portion 51 and the second operating portion 52 with physiological salt solution. In the initial state, the contact portion 30 is in a contracted state (i.e., not expanded or bent outwards), as depicted in FIGS. 1A and 2A. The inner tube 20 extending through the seal portion 54 is slidable relative to the seal portion 54.

To occlude a great saphenous vein or a small saphenous vein, normally an introducer sheath is inserted into the great saphenous vein or small saphenous vein by way of the knee. The knee provides easy access to the inside of the vein V. The medical device 1 in the initial state is then inserted through the introducer sheath into the vein V, starting from the distal end portion of the introducer sheath (insertion step). Note that the position at which to dispose the introducer sheath is not limited to the knee, and the insertion direction can be either an upstream direction or a downstream direction of the blood flow.

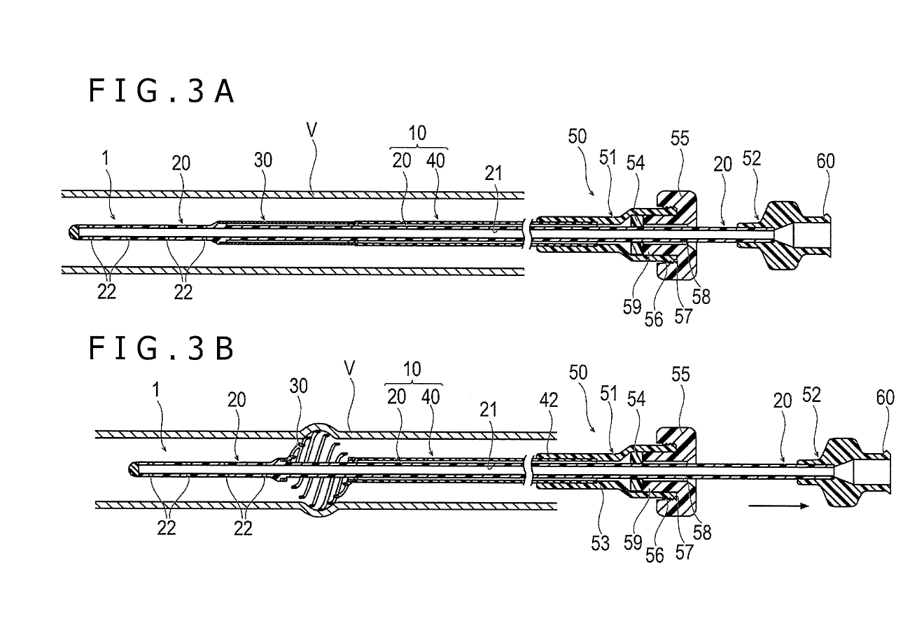

Next, the medical device 1 is pushed forward so that the contact portion 30 is pushed to the distal end of a treatment range for the vein V, as depicted in FIG. 3A. The distal end of the treatment range for the vein V may be, for example, in the vicinity of a joining portion of the superficial vein (e.g., the great saphenous vein or small saphenous vein) and a deep vein (e.g., a position deviated by 10 mm to 30 mm from the joining portion toward the superficial vein side).

Subsequently, the second operating portion 52 is moved proximally relative to the first operating portion 51 (or the first operating portion 51 is moved distally relative to the second operating portion 52). This relative movement causes the contact portion 30 to expand/bend outwardly. The contact portion 30 is expanded radially outwardly to contact the inner wall surface of the vein V, as depicted in FIG. 3B (contact step). After the contact portion 30 is expanded to an appropriate size, the seal adjusting portion 55 is rotated. Rotating the seal adjusting portion 55 causes the pressing portion 59 to move distally to compress the seal portion 54, thereby fixing the inner tube 20 in a non-slidable manner by the seal portion 54 (i.e., the seal portion 54 compresses against the inner tube 20 so that the inner tube 20 does not slide in the axial direction of the medical device 1 relative to the seal portion 54). By this operation, the expanded state of the contact portion 30 can be maintained (i.e., the contact portion 30 is held in the expanded state). In addition, when it is desired to increase the damage to the inner wall of the vein V, the outer diameter of the contact portion 30 may be further increased beyond the outer diameter necessary to contact the inner wall of the vein V. This further contact portion 30 diameter increase can be accomplished by releasing the seal adjusting portion 55 and moving the inner tube 20 proximally beyond its position at the expansion step. The deformation can be performed until the inner surface of the linear material portions 31 on the proximal side of the contact portion 30 and the inner surface of the linear material portions 31 on the distal side of the contact portion 30 come into contact with each other in the axial direction. By this process, the tops or peaks of the linear material portions 31 can be securely positioned in the inside of the inner wall surface of the vein V (i.e., the linear material portions 31 are pressed securely against the inner wall surface of the vein V), ensuring that twisting can be more easily performed and the vein inner wall can be more easily damaged.

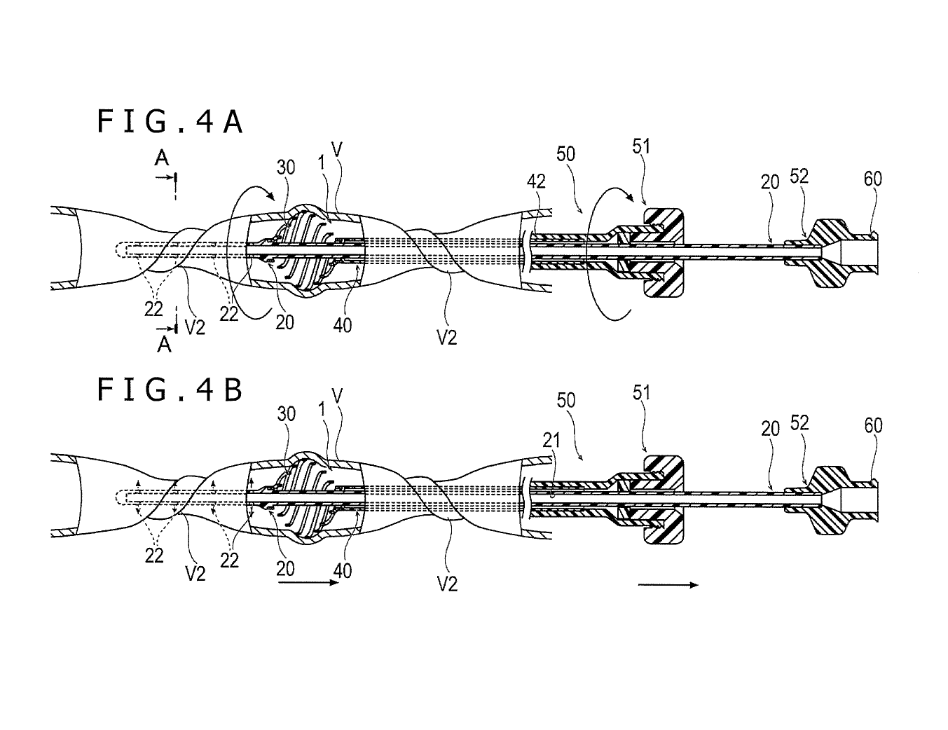

Next, the operating portion 50 is rotated, causing the contact portion 30 to rotate together with the inner tube 20 and the outer sheath 40. Due to the frictional resistance (i.e., the friction force) between the contact portion 30 and the inner wall of the vein V, the vein V rotates and twists as depicted in FIG. 4A (twisting step). By receiving a twisting force from the contact portion 30, the vein V is formed with twisted portions V2 (i.e., the locations when the vein V twists) on both sides of the contact portion 30, while being reduced in inside diameter at the twisted portions V2. At the twisted portion V2, at least one fold is formed on the circumference of the blood vessel, so that the inside diameter is reduced at that twisted portion V2.

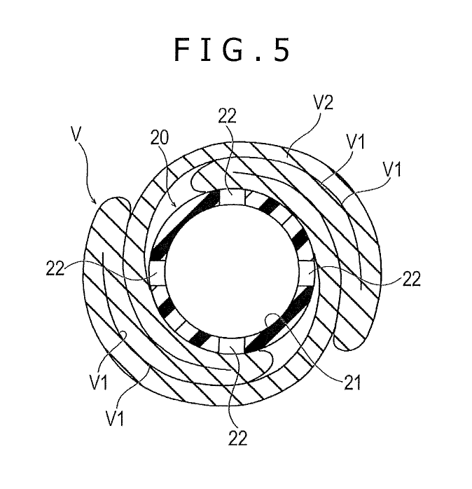

Note that the positions of the twisted portions V2 of the vein V and the degree of twisting of the twisted portions V2 vary depending on various conditions. Examples of conditions affecting the positions of the twisted portions V2 and the degree of twisting include the conditions of the vein V, the position of contact of the contact portion 30 with the vein V, etc. Depending on the various conditions, portions of an inner wall surface V1 (inner membrane) of the vein V at the twisted portion V2 can contact each other in an overlapping manner, as depicted in FIG. 5. When the inner wall surface V1 of the vein V is damaged, a pair of the inner membrane and a middle coat of the inner wall surface V1 of the vein V, a pair of the middle coat and the middle coat of the inner wall surface V1 of the vein V, a pair of the inner membrane and an outer membrane of the inner wall surface V1 of the vein V, a pair of the middle coat and the outer membrane of the inner wall surface V1 of the vein V, and/or a pair of the outer membrane and the outer membrane of the inner wall surface V1 of the vein V, can contact each other in an overlapping manner. In addition, at the twisted portion V2, the inner wall surface V1 of the vein V can make contact with the outer peripheral surfaces of the inner tube 20 and the outer sheath 40. This contact ensures that the blood flow in the vein V can be effectively blocked or reduced, thus reducing the amount of blood in the vein V. Note that portions of the inner wall surface V1 of the vein V may not contact each other, and the inner wall surface V1 of the vein V may not contact the outer peripheral surface of the inner tube 20 or the outer sheath 40 at the part where the inside diameter of the vein V is reduced. Even in such a situation, however, the twisting of the vein V makes it possible to reduce the blood flow in the vein V and to reduce the amount of blood in the vein V.

Subsequently, the entire body of the operating portion 50 is pulled to cause the contact portion 30 to move within the vein V while damaging the inner wall surface of the vein V (movement step). The inner wall surface of the vein V can be relatively evenly damaged over a wide range because the contact portion 30 has the spirally shaped linear material portions 31. In addition, the movement of the contact portion 30 within the vein V causes the twisted portions V2 of the vein V located on both sides of the contact portion 30 to move. The movement distance of the contact portion 30 is preferably within the length (for example, 100 mm) of the range L over which the inner tube side holes 22 are formed, that is, the range over which the treatment agent is released. Since the contact portion 30 is elastically deformable, the size of the contact portion 30 varies appropriately according to variations in the inside diameter of the vein V, so that the vein V can always be damaged suitably. The outer surface of the tip of the inner tube 20 can be coated with a lubricant such as a silicone oil for enhancing the ability of the tip to reach a peripheral portion in the blood vessel. The lubricant coating ensures that even when the blood vessel inside diameter is reduced upon twisting of the blood vessel and the blood vessel inner wall contacts the outer surface of the tip of the inner tube 20, the blood vessel inner wall will slide on the tip of the inner tube 20. There will thus be lower resistance (i.e., less friction force resisting the movement) between the blood vessel inner wall and the inner tube and moving the inner tube 20 becomes easier.

In addition, immediately after the insertion of the medical device 1 into the vein V, the contact portion 30 and the inner wall surface of the vein V may be brought into contact, and the entire body of the operating portion 50 may be pushed in (i.e., distally) from the insertion site to the distal end of the treatment range of the vein V. After the arrival of the operating portion 50 at the distal end, the entire body of the operating portion 50 may be rotated. By such a process, the contact portion 30 rotates together with the inner tube 20 and the outer sheath 40 as depicted in FIG. 4A, and, due to the frictional resistance between the contact portion 30 and the vein V, the vein V rotates to become twisted. The entire body of the operating portion 50 may then be pulled, whereby the inner wall surface of the vein V can be damaged because of the forward and backward movement of the operating portion 50 and thus the contact portion 30.

Subsequently, a syringe or the like filled with a treatment agent is connected to the injection port 60, and a predetermined amount of the treatment agent is injected. As depicted in FIG. 4B, the treatment agent thus flows within the first lumen 21 of the inner tube 20, and is released inside of the vein V through the inner tube side holes 22 (release step). As a result, the treatment agent comes into contact with the blood vessel wall (i.e., the inner wall of the vein V). If the treatment agent contacts the blood vessel wall for a predetermined immersion time, inflammation, thrombus formation or proliferation of smooth muscle cells will be induced in the blood vessel wall to effectively occlude or contract the blood vessel (e.g., vein V). Since the blood flow in the vein V is interrupted or reduced and the amount of blood in the vein V is reduced, the treatment agent flowing into the vein V is less likely to be carried away by the blood flow or to be diluted with blood. Therefore, diffusion of the treatment agent can be minimized so that the treatment agent acts on the blood vessel wall more effectively than when the amount of blood in the vein V is not reduced. This allows the vein V to be effectively occluded or contracted. Note that the treatment agent can arrive at the mutually contacting portions of the inner wall surface V1 at the twisted portions V2 of the vein V through the gap between the mutually contacting portions of the inner wall surface V1.

The twisting of the vein V may be canceled (i.e., the vein may become untwisted) when the contact portion 30 is gradually slid against the blood vessel wall. When the operating portion 50 is pulled, however, the movement of the spirally shaped linear material portions 31 in contact with the blood vessel wall ensures that, due to the inclination of the spiral of the linear material portions 31, a rotating force can be exerted on the vein V. This rotating force exerted on the vein V helps ensure that the twisted state of the vein V can be maintained. Therefore, the direction of the spiral of the linear material portions 31 is preferably a direction such that the rotating force exerted on the vein V supplements the twisting of the vein V when the contact portion 30 is moved. After the medical device 1 is moved by a distance corresponding to the range of the tip portion of the inner tube 20 over which the inner tube side holes 22 are formed, the medical device 1 is rotated in a direction opposite to the direction of twisting to cancel the twisting of the vein V (i.e., untwist the vein V). It is possible to cancel the twisting, then rotate the medical device 1 again in the direction for twisting the blood vessel and move the medical device 1 (i.e., untwisting, moving, and twisting can be performed in succession to act on different areas of the blood vessel).

Note that a process may be adopted in which the operation of rotating the operating portion 50 so as to twist the vein V is not conducted. Instead, only the pulling of the operating portion 50 is performed so that the inclination of the spiral of the linear material portions 31 making contact with the blood vessel wall causes a rotating force to act on the vein V, thereby twisting the vein V.

A predetermined amount of the treatment agent is then injected via the injection port 60 (as discussed above). This causes the treatment agent to flow into the first lumen 21 of the inner tube 20 and to be released through the inner tube side holes 22 into the inside of the vein V. As a result, the treatment agent acts effectively on the blood vessel wall physically damaged by the linear material portions 31, which, when introduced for a predetermined immersion time, induces inflammation, thrombus formation or proliferation of smooth muscle cells in the blood vessel wall or the like, whereby the vein V can be effectively occluded or contracted. Since the blood flow in the vein V is intercepted or reduced (i.e., blood flow in the vein V is stopped or reduced between the twisted portions V2) and the amount of blood in the vein V is reduced, the treatment agent flowing into the vein V is unlikely to be carried away by the blood flow and to be diluted with blood. Therefore, the treatment agent effectively acts on the damaged blood vessel wall, and the vein V can be effectively occluded or contracted.

Subsequently, the contact portion 30 may again be moved to damage the blood vessel wall, and the treatment agent is released via the injection port 60, to occlude or contract the vein V. Thereafter, the movement of the contact portion 30 and the release of the treatment agent are repeated, whereby the vein V in a desired range can be entirely occluded or contracted.

After the treatment of the desired range of the vein V is finished, the operating portion 50 is rotated in a direction for canceling the twisting of the twisted portions V2 (i.e., untwisting the twisted portions V2). By this operation, the twisting of the twisted portions V2 is canceled. Next, the seal adjusting portion 55 is rotated to move the pressing portion 59 proximally, whereby the compression of the seal portion 54 is weakened (i.e., the pressing portion 59 moves proximally to remove the compression force against the seal portion 54) and the inner tube 20 penetrating the seal portion 54 is made slidable. Thereafter, when the second operating portion 52 is moved distally relative to the first operating portion 51 (or the first operating portion 51 is moved proximally relative to the second operating portion 52), the contact portion 30 contracts, and the medical device 1 returns into the initial state depicted in FIG. 3A.

The medical device 1 is then drawn out of the introducer sheath, and the introducer sheath is drawn out of the vein V, to complete the procedure.

As has been described above, the blood vessel treatment method according to the first embodiment includes: (i) the insertion step of inserting into a blood vessel the medical device provided with the elongate shaft portion and the contact portion capable of contacting a biological tissue in the blood vessel; (ii) the contact step of bringing the contact portion into contact with the biological tissue in the blood vessel; (iii) the twisting step of twisting the blood vessel by the contact portion; and (iv) the release step of releasing from the medical device the treatment agent for occluding or contracting the lumen of the blood vessel. The blood vessel treatment method configured as above ensures that the treatment agent can be released in a condition where the blood flow in the blood vessel is interrupted or reduced and the amount of blood in the blood vessel is reduced, owing to the twisting of the blood vessel. Therefore, the treatment agent flowing into the vein is less liable to be carried away by the blood flow and to be diluted with blood. Consequently, diffusion of the treatment agent can be restrained, the treatment agent can be made to effectively act on the blood vessel wall, and the blood vessel can be effectively occluded or contracted.

The contact portion may be expanded to the radially outer side of the shaft portion so as to contact the biological tissue. Therefore, the contact portion can be delivered to a target part of a blood vessel in its contracted state before expansion. Thus, enhanced operability is realized.

The blood vessel treatment method described above may further include the movement step of moving the contact portion in the axial direction when the contact portion makes contact with the biological tissue to damage the biological tissue. The biological tissue can thus be damaged efficiently. In addition, the positions at which the blood vessel is twisted (e.g., twisted portions V2) are moved following up to the movement of the contact portion, so that the contact portion can always be disposed in a region where the blood flow in the blood vessel is cut off or reduced and where the amount of blood is reduced. Therefore, even where a wide range of the blood vessel is to be treated, the treatment agent can be made to act effectively on the blood vessel wall while the contact portion is being moved. Consequently, the lumen of the blood vessel can be effectively occluded or contracted.

The contact portion having the spirally shaped linear material portions may be put into contact with the biological tissue of a blood vessel. In the twisting step, the contact portion moves in the axial direction of the shaft portion so as to impart a rotating force to the blood vessel by the contact portion, thereby twisting the blood vessel. Therefore, the blood vessel can be twisted by utilizing the axial movement of the contact portion, which ensures enhanced operability (i.e., operability may be easier because only axial movement is required to twist the vein V).

The inside diameter of the blood vessel is reduced (i.e., the inner wall surfaces of the blood vessel move closer or contract) by being twisted. The insider diameter of the blood vessel may be sufficiently reduced so that the inner wall surface of the blood vessel is brought into contact with the shaft portion, so that the blood flow path inside the blood vessel can be reduced effectively. Therefore, the treatment agent can be released into the blood vessel in a condition where the blood flow and the amount of blood in the blood vessel are effectively reduced. Accordingly, diffusion of the treatment agent can be restrained, and the treatment agent can be made to act effectively on the blood vessel wall. As a consequence, the blood vessel can be effectively occluded or contracted.

The inner wall surface of the blood vessel reduced in inside diameter by being twisted is brought into contact with the part of the shaft portion that has openings for releasing the treatment agent. Therefore, the treatment agent can be made to act effectively on the blood vessel wall while the flow path inside the blood vessel is being reduced effectively. Consequently, the blood vessel can be effectively occluded or contracted.

Portions of the inner wall surface of the twisted blood vessel may be brought into contact with each other in an overlapping manner. Therefore, the treatment agent can be made to act effectively on the blood vessel wall while the flow path inside the blood vessel is being effectively reduced. As a result, the blood vessel can be effectively occluded or contracted.

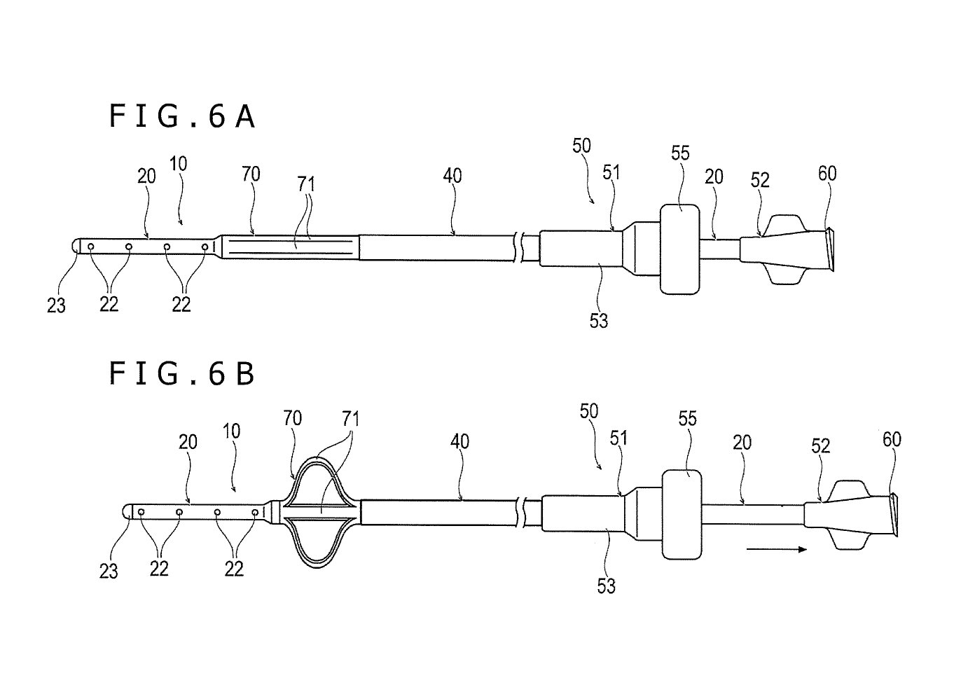

The configuration of the contact portion is not specifically restricted or limited to the configuration described above, so long as the contact portion can be expanded and contracted by relative movements of the outer sheath 40 and the inner tube 20. For example, in a modification of the first embodiment illustrated in FIGS. 6A and 6B, a contact portion 70 may have rectilinearly shaped linear (filamentous) material portions 71, instead of the spirally shaped linear (filamentous) material portions. The vein V can be twisted by the contact portion 70 with the rectilinearly shaped linear material portions, and the vein V can be damaged by moving the contact portion 70.

A medical device 80 according to a second embodiment differs from the first embodiment in that a treatment agent such as a sclerosant or an adhesive is released on a proximal side of a contact portion 30. The components or parts having functions equivalent or similar to those in the first embodiment are denoted by the same reference numerals as used above, and description of those parts is omitted.

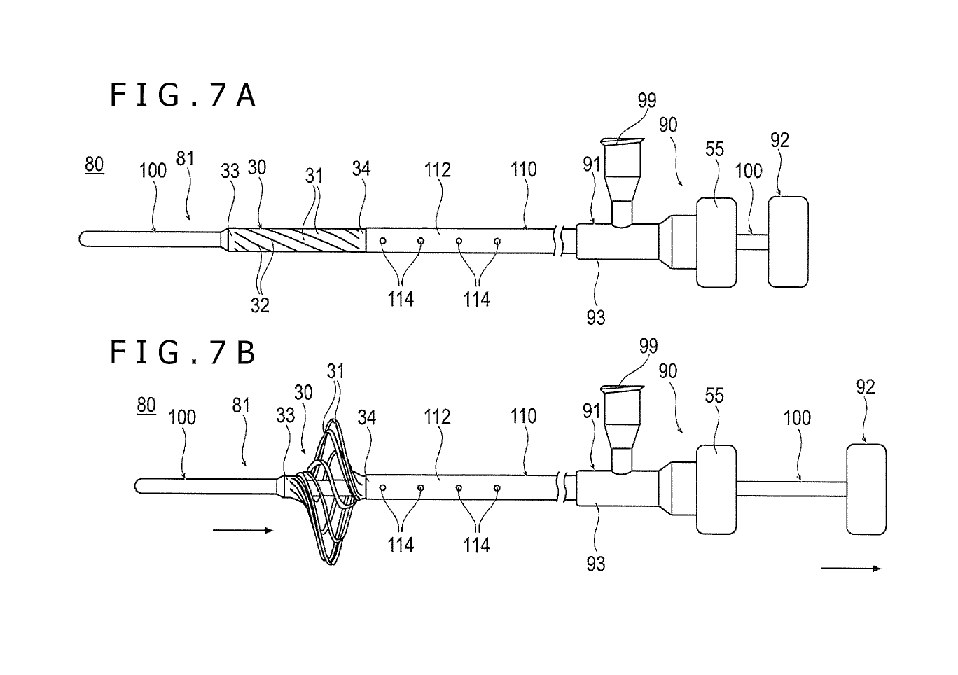

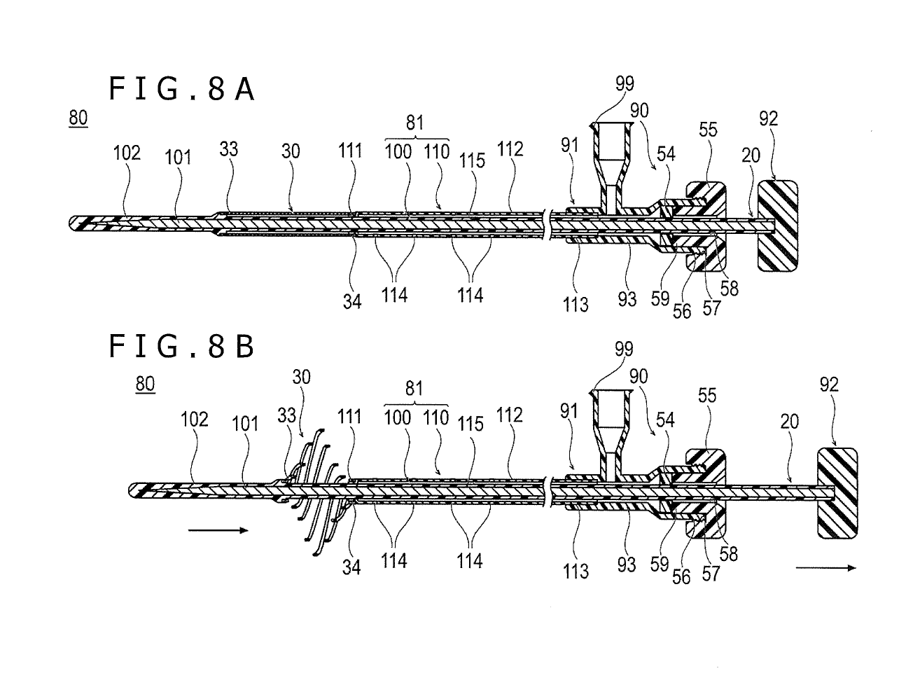

The medical device 80 depicted in FIGS. 7A to 8B includes an elongate shaft portion 81, a contact portion 30 capable of expansion and contraction on a distal side of the shaft portion 81, and an operating portion 90 for operating the contact portion 30. The shaft portion 81 includes an elongate inside shaft portion 100, and a pipe shaped outer sheath 110 accommodating the inside shaft portion 100 (i.e., the outer sheath 110 surrounds the inside shaft portion 100).

The inside shaft portion 100 includes a core member 101 for imparting desired rigidity (stiffness), and a cover body 102 covering (i.e., surrounding) an outer peripheral surface of the core member 101. A distal portion of the core member 101 is gradually reduced in diameter along the distal direction (i.e., the distal portion of the core member 101 is tapered in the distal direction) and has a gradient physical property of becoming more flexible on more distal side, for the purpose of reducing its influence on a biological tissue of the vein V. The gradient physical property here refers to a property of having a rigidity gradually lowered along the direction from the proximal side toward the distal side of the medical device 80 (i.e., the distal portion of the core member 101 increases in flexibility in the distal direction). A proximal portion of the inside shaft portion 100 is fixed to a second operating portion 92 of the operating portion 90. The operating portion 90 includes the first operating portion 91 and the second operating portion 92.

The outer sheath 110 is a pipe body accommodating the inside shaft portion 100 (i.e., surrounding the inside shaft portion 100 so that the inside shaft portion 100 is within the outer sheath 110), and is movable in an axial direction relative to the inside shaft portion 100. A distal end portion 111 of the outer sheath 110 is slidable in close contact with an outer peripheral surface of the inside shaft portion 100. A proximal end portion 113 of the outer sheath 110 is connected to the first operating portion 91 of the operating portion 90. The distal end portion 111 of the outer sheath 110 is formed with an outer sheath main body portion 112 greater in inside diameter than the distal end portion 111 so that a predetermined gap is formed between the outer sheath main body portion 112 and an outer peripheral surface of the inside shaft portion 100. The outer sheath main body portion 112 is formed with a plurality of outer sheath side holes 114 piercing from an inner surface to an outer surface of the outer sheath main body portion 112 (i.e., the outer sheath side holes 114 are holes through the wall of the outer sheath main body portion 112). The outer sheath side holes 114 are aligned in the axial direction and in the circumferential direction. A second lumen 115 is positioned between the outer sheath 110 and the inside shaft portion 100. A treatment agent can be introduced into and flow within the second lumen 115. The treatment agent flowing into the second lumen 115 can flow out through the outer sheath side holes 114 to the exterior.

The distal end portion of the contact portion 30 is secured to a distal portion of the inside shaft portion 100, and it's the proximal end portion 34 of the contact portion 30 is secured to a distal portion of the outer sheath 110. The contact portion 30 includes a plurality of spirally shaped linear material portions 31 aligned in the circumferential direction of the contact portion 30. Each of the linear material portions 31 can be bend outward to expand and come away (i.e., move radially away) from the outer peripheral surface of the inside shaft portion 100. The linear material portions 31 are expanded outward by proximally moving the inside shaft portion 100 relative to the outer sheath 110 (see FIGS. 7B and 8B). From the expanded state, each linear material portion 31 can be contracted to come closer to the outer peripheral surface of the inside shaft portion 100 by distally moving the inside shaft portion 100 relative to the outer sheath 110 (see FIGS. 7A and 8A).

The material constituting the core member 101 is preferably a hard, flexible material. Examples include shape memory alloys to which a shape memory effect or superelasticity is imparted by heat treatment, stainless steel, Ta, Ti, Pt, Au, W, and so on. Preferable examples of the shape memory alloys include Ni—Ti alloys, Cu—Al—Ni alloys, and Cu—Zn—Al alloys.

The material constituting the cover body 102 is preferably a hard, flexible material. Examples include polyolefins such as polyethylene, polypropylene, etc., polyamides, polyesters such as polyethylene terephthalate, etc., fluoropolymers such as ETFE, etc., PEEK, polyimides, and the like.

The operating portion 90 includes the first operating portion 91 connected to the proximal end portion 113 of the outer sheath 110, and the second operating portion 92 connected to the proximal end portion of the inside shaft portion 100. The first operating portion 91 includes an operating portion main body 93 fitted and connected to the proximal end portion 113 of the outer sheath 110, a seal portion 54 inside of the operating portion main body 93, and a seal adjusting portion 55 connected to the proximal end portion of the operating portion main body 93.

The operating portion main body 93 is a pipe shaped member. The proximal end portion of the inside shaft portion 100 is fitted and connected to the inside on the distal side of the operating portion main body 93, and the seal portion 54 is disposed on the inside on the proximal side of the operating portion main body 93. The outer peripheral surface of the operating portion main body 93 is formed with a male screw portion 56 for screw engagement with a female screw portion 57 of the seal adjusting portion 55. In addition, the operating portion main body 93 is provided with an injection port 99 which opens sideways and through which a treatment agent can be injected into the inside of the operating portion main body 93. A three-way cock or a syringe or the like can be connected to the injection port 99.

The second operating portion 92 is connected to the proximal portion of the inside shaft portion 100.

When the second operating portion 92 is moved proximally relative to the first operating portion 91, the distal end portion 33 and the proximal end portion 34 of the contact portion 30 move closer to one another, and the linear material portions 31 expand while bending outward so as to move radially away from the outer peripheral surface of the inside shaft portion 100 (see FIGS. 7B and 8B). When the second operating portion 92 is moved distally relative to the first operating portion 91, the distal end portion 33 and the proximal end portion 34 of the contact portion 30 move away from each other, and the linear material portions 31 contract and move closer to the outer peripheral surface of the inside shaft portion 100 (see FIGS. 7A and 8A).

The materials constituting the operating portion main body 93 and the second operating portion 92 are not particularly limited. Examples include rigid resins such as polycarbonate, polyamides, polypropylene, etc.

A method of using the medical device 80 according to the second embodiment is described below, in reference to an example of occluding a vein V in the state of varicose vein occurring in a great saphenous vein or a small saphenous vein of a lower limb.

First, the medical device 80 to be used is primed by flushing the inside of the contact portion 30, the outer sheath 110 and the first operating portion 91 with physiological salt solution. In the initial state, as depicted in FIGS. 7A and 8A, the contact portion 30 is in a contracted state (i.e., not expanded or bent outwards). The inside shaft portion 100 penetrating the seal portion 54 is slidable relative to the seal portion 54.

To occlude a great saphenous vein or a small saphenous vein, normally an introducer sheath is inserted into the great saphenous vein or small saphenous vein by way of the knee. The knee provides easy access to the inside of the vein V. The medical device 80 in the initial state is then inserted through the introducer sheath into the vein V, starting from the distal end portion of the introducer sheath (insertion step).

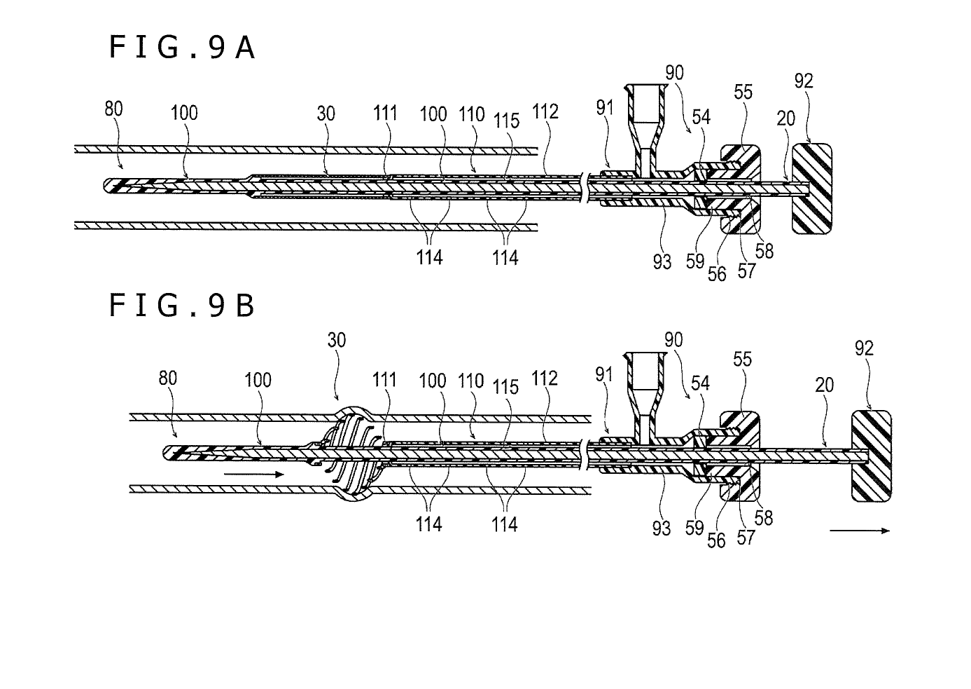

Next, the medical device 80 is pushed forward so that the contact portion 30 is pushed to the distal end of a treatment range for the vein V, as depicted in FIG. 9A.

Subsequently, the second operating portion 92 is moved proximally relative to the first operating portion 91 (or the first operating portion 91 is moved distally relative to the second operating portion 92). This relative movement causes the contact portion 30 to expand/bend outwardly. The contact portion 30 is expanded radially outwardly, as depicted in FIG. 9B, to contact an inner wall surface of the vein V (contact step). After the contact portion 30 is expanded to an appropriate size, the seal adjusting portion 55 is rotated. Rotating the seal adjusting portion 55 causes the pressing portion 59 to move distally to compress the seal portion 54, and the inside shaft portion 100 is fixed in a non-slidable manner by the seal portion 54 (i.e., the seal portion 54 compresses against the inner tube 20 so that the inner tube 20 does not axially slide relative to the seal portion 54). As a result, the expanded state of the contact portion 30 can be maintained (i.e., the contact portion 30 is fixed in the expanded state).

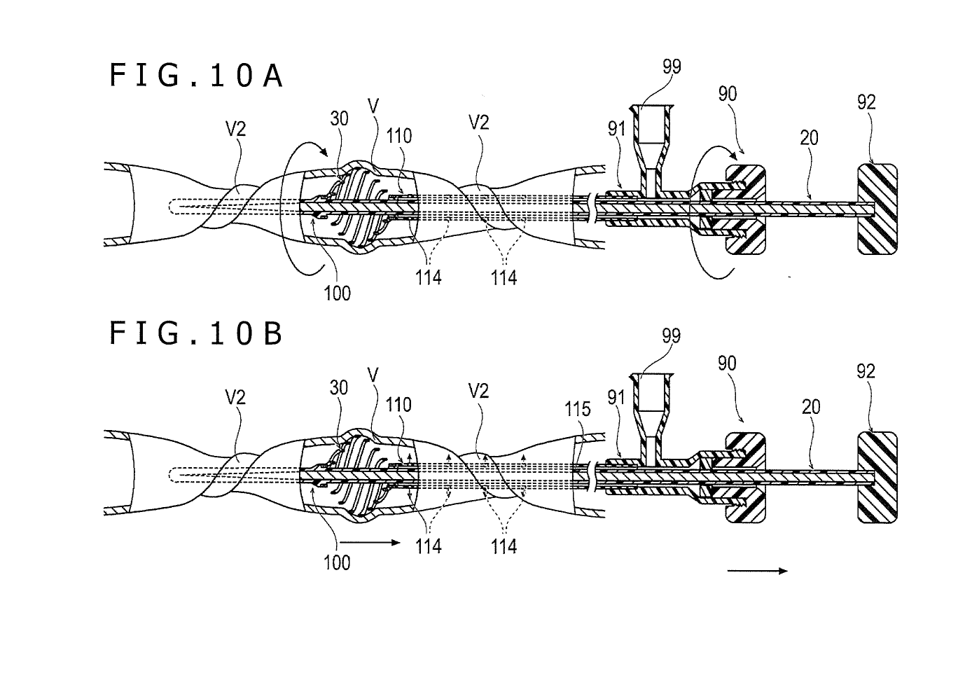

Next, the entire body of the operating portion 90 is rotated, causing the contact portion 30 to rotate together with the inside shaft portion 100 and the outer sheath 110 as depicted in FIG. 10A. Due to the frictional resistance between the contact portion 30 and the inner wall of the vein V, the vein V rotates and twists (twisting step). By receiving a twisting force from the contact portion 30, the vein V is formed with twisted portions V2, and reduced in inside diameter, on both sides of the contact portion 30.

Subsequently, a syringe or the like filled with a treatment agent is connected to the injection port 99, and a predetermined amount of the treatment agent is injected. As depicted in FIG. 10B, the treatment agent is permitted to flow into the second lumen 115 of the outer sheath 110, to be released through the outer sheath side holes 114 into the inside of the vein V (release step). As a result, the treatment agent contacts the blood vessel wall. If the treatment agent contacts the blood vessel wall for a predetermined immersion time inflammation, thrombus formation or proliferation of smooth muscle cells is induced in the blood vessel wall or the like, whereby the vein V can be effectively occluded or contracted. The blood flow in the vein V is thus interrupted or reduced, and the amount of blood in the vein V is reduced. In this state, the treatment agent flowing into the vein V is less likely to be carried away by the blood flow and to be diluted with blood. Accordingly, diffusion of the treatment agent can be restrained, and the treatment agent is made to act effectively on the blood vessel wall. As a consequence, the vein V can be effectively occluded or contracted.

Next, the entire body of the operating portion 90 is pulled to cause the contact portion 30 to move within the vein V while damaging the inner wall surface of the vein V (movement step). Movement of the contact portion 30 within the vein V causes the twisted portion V2 of the vein V located on both sides of the contact portion 30 to also move.

The twisting of the vein V is liable to be gradually canceled (i.e., the vein untwists) by sliding between the contact portion 30 and the blood vessel wall. When the operating portion 90 is pulled, however, the spirally shaped linear material portions 31 move while contacting the blood vessel wall to exert a rotating force on the vein V due to the inclination of spiral of the linear material portions 31, whereby the twisted state of the vein V can be maintained.

Subsequently, a predetermined amount of the treatment agent is again injected via the injection port 99. By this operation, the fluid is permitted to flow into the second lumen 115 of the outer sheath 110, to be released through the outer sheath side holes 114 into the inside of the vein V. As a result, the treatment agent acts effectively on the blood vessel wall physically damaged by the linear material portions 31. When the treatment agent is introduced for a predetermined immersion time, inflammation, thrombus formation or proliferation of smooth muscle cells will be induced in the blood vessel wall or the like. This immersion effectively occludes or contracts the vein V. In this instance, the blood flow in the vein V is intercepted or reduced, and the amount of blood in the vein V is reduced. In this state, therefore, the treatment agent flowing into the vein V is less liable to be carried by the blood flow and be diluted with blood. For this reason, the treatment agent can be made to act effectively on the damaged blood vessel wall. Consequently, the vein V can be effectively occluded or contracted.

Next, the contact portion 30 is again moved to damage the blood vessel wall, and the treatment agent is released via the injection port 99, to occlude or contract the vein V. Thereafter, the movement of the contact portion 30 and the release of the treatment agent are repeated, whereby the entire body of the vein V in a desired range can be occluded.

After the treatment of the vein V in the desired range is finished, the operating portion 90 is rotated in a direction for canceling the twisting of the twisted portions V2. By this operation, the twisting of the twisted portions V2 is canceled (i.e., the vein V is untwisted). Next, the seal adjusting portion 55 is rotated to move the pressing portion 59 proximally and to weaken the compression of the seal portion 54 (i.e., the seal portion 54 becomes less compressed), whereby the inside shaft portion 100 penetrating the seal portion 54 is made slidable. The second operating portion 92 is then moved distally relative to the first operating portion 91 (or the first operating portion 91 is moved proximally relative to the second operating portion 92) causing the contact portion 30 to contract, and the medical device 80 to return to the initial state depicted in FIG. 9A.

The medical device 80 is then drawn out of the introducer sheath, and the introducer sheath is drawn out of the vein V, to complete the procedure.

Thus, the medical device 80 according to the second embodiment releases the treatment agent on the proximal side of the contact portion 30, unlike in the first embodiment. The treatment agent can be released in the blood vessel in this configuration while the blood vessel is in the state of being twisted by the contact portion 30. Therefore, diffusion of the treatment agent can be restrained, and the treatment agent can be made to act effectively on the blood vessel wall. Consequently, the blood vessel can be effectively occluded or contracted.

A medical device 120 according to a third embodiment differs from the first and second embodiments in that a contact portion is configured by use of a bent linear material. Note that parts equivalent or similar to those in the first and second embodiments are denoted by the same reference numerals as used above, and description of those parts is omitted.

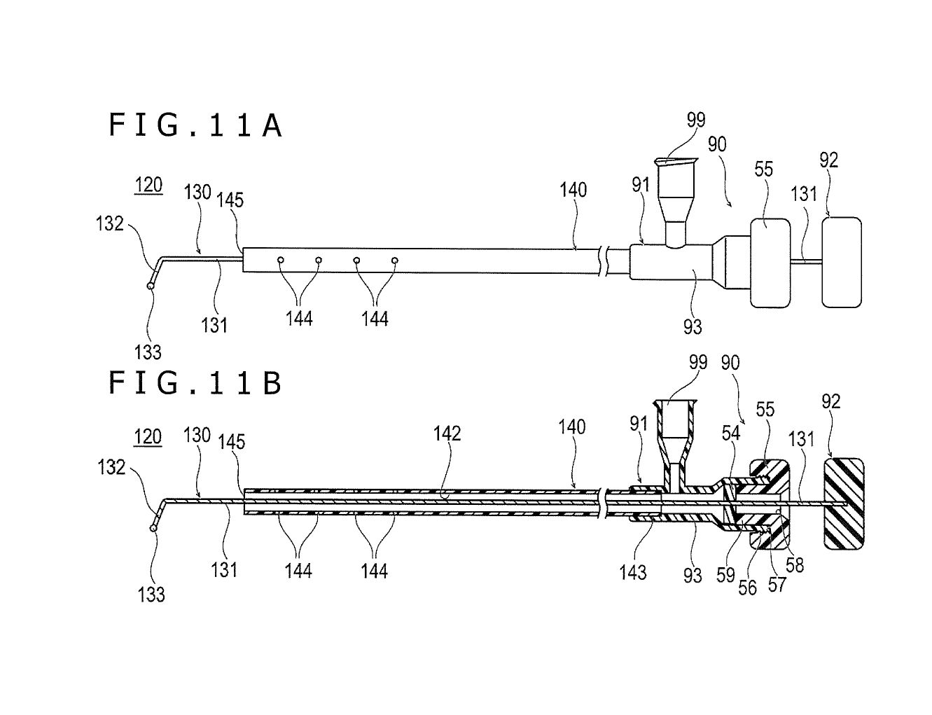

The medical device 120, as depicted in FIGS. 11A and 11B, includes an elongate shaft portion 130, a contact portion 132 capable of expansion (i.e., configured to expand) to the radially outer side of the shaft portion 130 at a distal portion of the shaft portion 130, and an operating portion 90 for operating the contact portion 132. The shaft portion 130 includes an elongate inside shaft portion 131, and a pipe shaped outer sheath 140 accommodating (i.e., surrounding) the inside shaft portion 131.

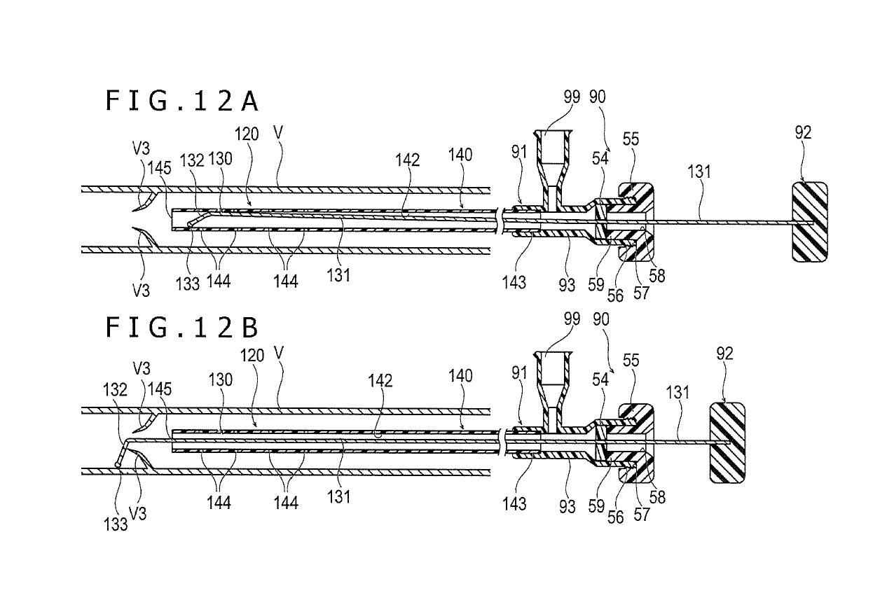

The inside shaft portion 131 is a linear member that is integral with the contact portion 132. The contact portion 132 is bent at an angle of less than 90 degrees relative to the inside shaft portion 131, which extends substantially rectilinearly (i.e., the contact portion 132 is bent less than 90 degrees so that the angle between the contact portion 132 and the inside shaft portion 131 is greater 90 degrees as shown, for example, in FIGS. 11A and 11B). The contact portion 132 has a spherically shaped tip contact portion 133 at its distal end portion set greater in outside diameter than the linear portion. The spherically shaped tip contact portion 133 reduces the influence exerted on a biological tissue. The second operating portion 92 is fixed to a proximal portion of the inside shaft portion 131. The contact portion 132, by protruding distally from the outer sheath 140, comes into a bent expanded state (see FIG. 11B), and, by being accommodated into the outer sheath 140, comes into a contracted state of being elastically deformed so that the bent portion approaches a rectilinear form (see FIG. 12A). In other words, the contact portion 132 becomes more bent or is further from a rectilinear shape when the contact portion 132 protrudes externally of the outer sheath 140 than when the contact portion 132 is within the outer sheath 140.

The outer sheath 140 is a pipe body accommodating the inside shaft portion 131, and is movable in an axial direction relative to the inside shaft portion 131. A proximal end portion 143 of the outer sheath 140 is connected to a first operating portion 91 of the operating portion 90. The inside diameter of the outer sheath 140 is greater than the outside diameter of the inside shaft portion 131 so that a gap is formed between the outer sheath 140 and an outer peripheral surface of the inside shaft portion 131. The outer sheath 140 is formed with a plurality of outer sheath side holes 144 piercing from an inner surface to an outer surface of the outer sheath 140 (i.e., the outer sheath side holes 144 are holes through the wall of the outer sheath 140), the outer sheath side holes 144 being aligned in the axial direction and aligned in the circumferential direction. Note that the outer sheath side holes 144 may not necessarily be included. A second lumen 142 is between the outer sheath 140 and the inside shaft portion 131 through which a treatment agent can flow. The fluid flowing into the second lumen 142 can flow out through the outer sheath side holes 144 (if included) and through a tip opening 145 of the outer sheath 140 to the exterior.

The materials constituting the inside shaft portion 131, the contact portion 132 and the outer sheath 140 are preferably hard, flexible materials. Examples include polyolefins such as polyethylene, polypropylene, etc., polyamides, polyesters such as polyethylene terephthalate, etc., fluoropolymers such as ETFE, etc., PEEK, polyimides, shape memory alloys to which a shape memory effect or superelasticity is imparted by heat treatment, stainless steel, Ta, Ti, Pt, Au, W, and so on. Examples of preferably usable shape memory alloys include Ni—Ti alloys, Cu—Al—Ni alloys, Cu—Zn—Al alloys, and so on.

The operating portion 90 includes the first operating portion 91 connected to the proximal end portion 143 of the outer sheath 140, and the second operating portion 92 connected to a distal end portion of the inside shaft portion 131. The first operating portion 91 includes an operating portion main body 93 fitted and connected to the proximal end portion 143 of the outer sheath 140, a seal portion 54 disposed inside the operating portion main body 93, and a seal adjusting portion 55 connected to the proximal end portion of the operating portion main body 93.

The operating portion main body 93 is a pipe shaped member. The proximal end portion of the inside shaft portion 131 is fitted and connected to the inside of the operating portion main body 93 on the distal side of the operating portion main body 93. The seal portion 54 is disposed on the inside of the proximal side of the operating portion main body 93. The outer peripheral surface on the proximal side of the operating portion main body 93 is formed with a male screw portion 56 for engagement with the seal adjusting portion 55. In addition, the operating portion main body 93 is provided with an injection port 99 which opens sideways. The treatment agent can be injected into the inside of the operating portion main body 93 through the injection port 99. The second operating portion 92 is connected to the proximal portion of the inside shaft portion 131.

Now, a method of using the medical device 120 according to the third embodiment is described below, in reference to an example of occluding a vein V in the state of a varicose vein occurring in a great saphenous vein or a small saphenous vein of a lower limb.

First, the medical device 120 to be used is primed by flushing the inside of the outer sheath 140 and the first operating portion 91 with physiological salt solution. In this initial state, the contact portion 132 is in a contracted state while being accommodated in the outer sheath 140 (see FIG. 12A). The inside shaft portion 131 penetrating the seal portion 54 is slidable relative to the seal portion 54.

To occlude a great saphenous vein or a small saphenous vein, normally an introducer sheath is inserted into the great saphenous vein or small saphenous vein by way of the knee. The knee provides easy access to the inside of the vein V. Thereafter, the medical device 120 prepared in the initial state is inserted through the introducer sheath into the vein V, starting from a distal end portion of the introducer sheath (insertion step).

Next, the medical device 120 is pushed forward so that the contact portion 132 is pushed to the distal end of a treatment range for the vein V, as depicted in FIG. 12A.

Subsequently, the second operating portion 92 is moved distally relative to the first operating portion 91 (or the first operating portion 91 is moved proximally relative to the second operating portion 92). Upon this operation, as depicted in FIG. 12B, the contact portion 132 protrudes from the tip opening 145 of the outer sheath 140, and the contact portion 132 returns into a bent state under its own restoring force (i.e., the contact portion 132 is in a bent state when no external force is applied, and the restoring force of the contact portion 132 returns the contact portion 132 to this bent state when the contact portion 132 protrudes from the tip opening 145 of the outer sheath 140), coming into a radially outwardly expanded state.

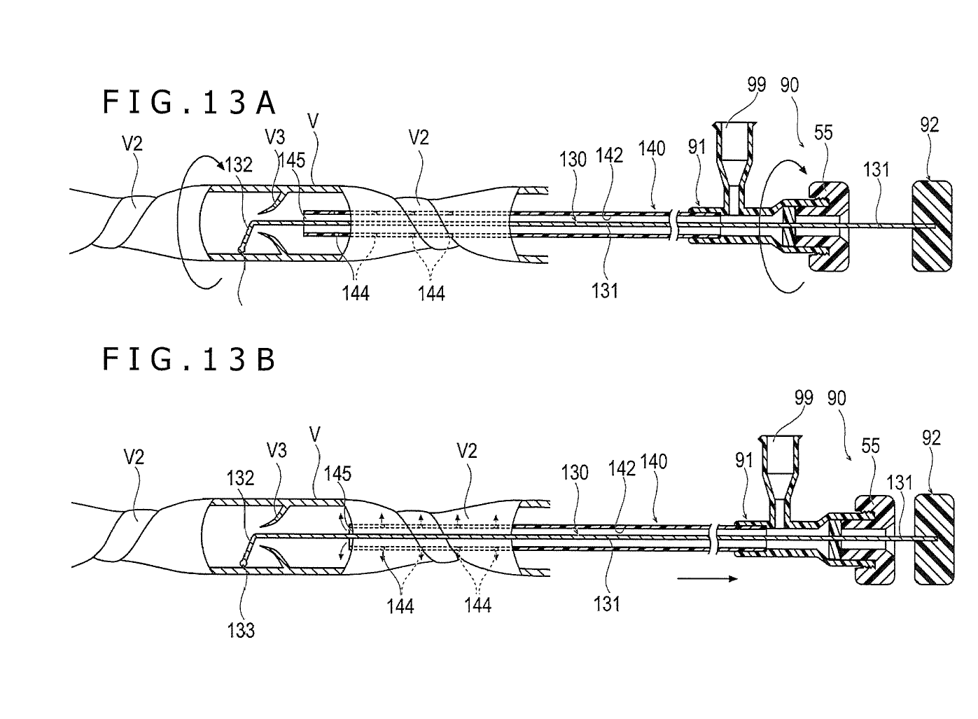

Next, the contact portion 132 is disposed between a pair of venous valves V3 (valve) which are disposed oppositely within the vein V, and is caught on or contacts with the venous valve V3 (contact step). After the contact portion 132 is caught on the venous valve V3 in the contact step, the second operating portion 92 or the entire body of the operating portion 90 is rotated. By this operation, as depicted in FIG. 13A, the contact portion 132 rotates together with the inside shaft portion 131, and the venous valve V3 receives a rotating force exerted by the contact portion 132, so that the vein V rotates and twists (twisting step). The vein V receives the twisting force exerted by the contact portion 132 and forms two twisted portions V2 on both sides of the contact portion 132. The twisted portions V2 have a reduced inside diameter than the other portions of the vein V.

Subsequently, a syringe or the like filled with a treatment agent is connected to the injection port 99, and a predetermined amount of the treatment agent is injected. As depicted in FIG. 13B, the treatment agent flows into the second lumen 142 of the outer sheath 140 and is released out of the outer sheath 140 through the outer sheath side holes 144 and through the tip opening 145 into the inside of the vein V (release step). As a result, the treatment agent contacts the blood vessel wall. If the treatment agent contacts the blood vessel wall for a predetermined immersion time, inflammation, thrombus formation or proliferation of smooth muscle cells will be induced in the blood vessel wall, whereby the vein V can be effectively occluded or contracted. In this instance, the blood flow in the vein V is interrupted or reduced, and the amount of blood in the vein V is reduced. Therefore, the treatment agent flowing into the vein V is less liable to be carried away by the blood flow and be diluted with blood. Accordingly, diffusion of the treatment agent can be restrained, and the treatment agent can be made to act effectively on the blood vessel wall. Consequently, the vein V can be effectively occluded or contracted.

Next, with the position of the second operating portion 92 maintained, the first operating portion 91 is moved proximally (movement step). By this operation, while the contact portion 132 provided at a tip portion of the inside shaft portion 131 connected to the second operating portion 92 is kept unmoved (i.e., held in place), the outer sheath 140 capable of releasing the treatment agent is moved proximally.

Subsequently, a predetermined amount of the treatment agent is again injected via the injection port 99, and the contact portion 132 is moved within the fluid release range to damage the blood vessel, thereby causing the vein V to be occluded or contracted. Thereafter, the movement of the outer sheath 140 and the release of the treatment agent are repeated, whereby the vein V in a desired range can be entirely occluded or contracted.

After the treatment of the desired range of the vein V is finished, the operating portion 90 is rotated in a direction for canceling the twisting of the twisted portions V2 (i.e., untwisting the twisted portions V2). By this operation, the twisting of the twisted portions V2 is canceled. Next, the second operating portion 92 is moved distally relative to the first operating portion 91 (or the first operating portion 91 is moved proximally relative to the second operating portion 92) to cause the contact portion 132 to be accommodated into the outer sheath 140 while being contracted (i.e., the contact portion 132 moves proximally to move within the outer sheath 140, and the bent angle of the contact portion is reduced to a more rectilinear shape or a shape that is closer to extending in the axial direction of the medical device 120), and the medical device 120 is returned into the initial state depicted in FIG. 12A.

Thereafter, the medical device 120 is drawn out of the introducer sheath, and the introducer sheath is drawn out of the vein V, to complete the procedure.

As has been described above, according to the medical device 120 of the third embodiment, the treatment agent can be released in the blood vessel that is being twisted by the contact portion 132. Therefore, the treatment agent can be made to act effectively on the blood vessel wall, so that the lumen of the blood vessel can be effectively occluded or contracted.

In the blood vessel treatment method according to the third embodiment, the contact portion 132 engages with the venous valve V3 (valve) of the blood vessel in the twisting step. Therefore, a rotating force can be easily exerted on the blood vessel from the contact portion 132, so that the blood vessel can be twisted effectively.

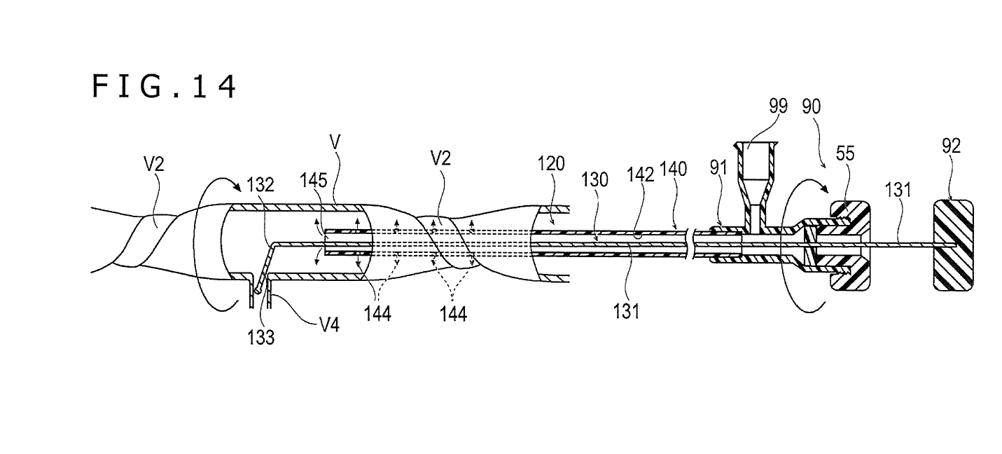

The contact portion 132 of the medical device 120 according to the third embodiment may be caught on another blood vessel component and is not limited to the venous valve V3. For example, as depicted in FIG. 14, the contact portion 132 may be inserted into a branch portion V4. A branch portion V4 is a blood vessel branched from the vein V, and the medical device 120 may be inserted into the branch portion V4 and twisted, to twist the vein V in this condition.

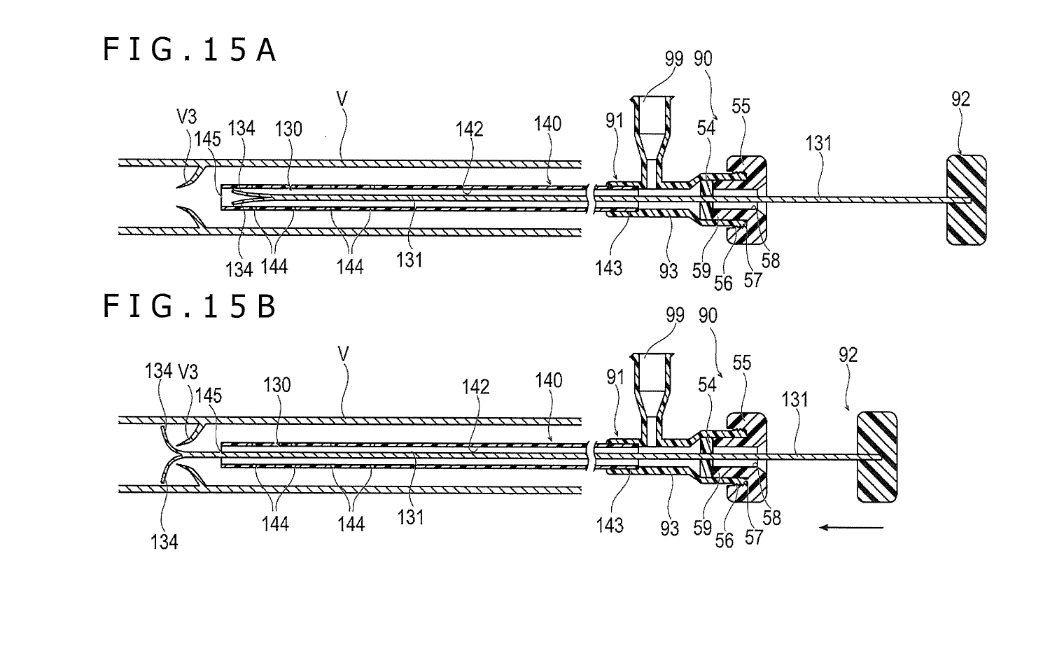

As illustrated by a first modification of the third embodiment in FIGS. 15A and 15B, a contact portion 134 may be formed to be bifurcated from a distal portion of the inside shaft portion 131. Note that parts having functions equivalent or similar to those in the third embodiment are denoted by the same reference signs as used above, and description of those parts is omitted. The contact portion 134, as depicted in FIG. 15A, can be accommodated in the outer sheath 140 while being elastically deformed. Further, when the second operating portion 92 is moved distally relative to the first operating portion 91 to protrude the contact portion 134 distally from the outer sheath 140, the contact portion 134 is expanded in the manner of being bifurcated wider by its own restoring force (i.e., when no external force is exerted on the contact portion 134, the contact portion 134 expands more radially outwards than when the contact portion 134 is within the outer sheath 140). The bifurcated contact portion 134 may engage with the venous valve V3 or with the branch portion V4. When the operating portion 90 is subsequently rotated, the vein V twists.

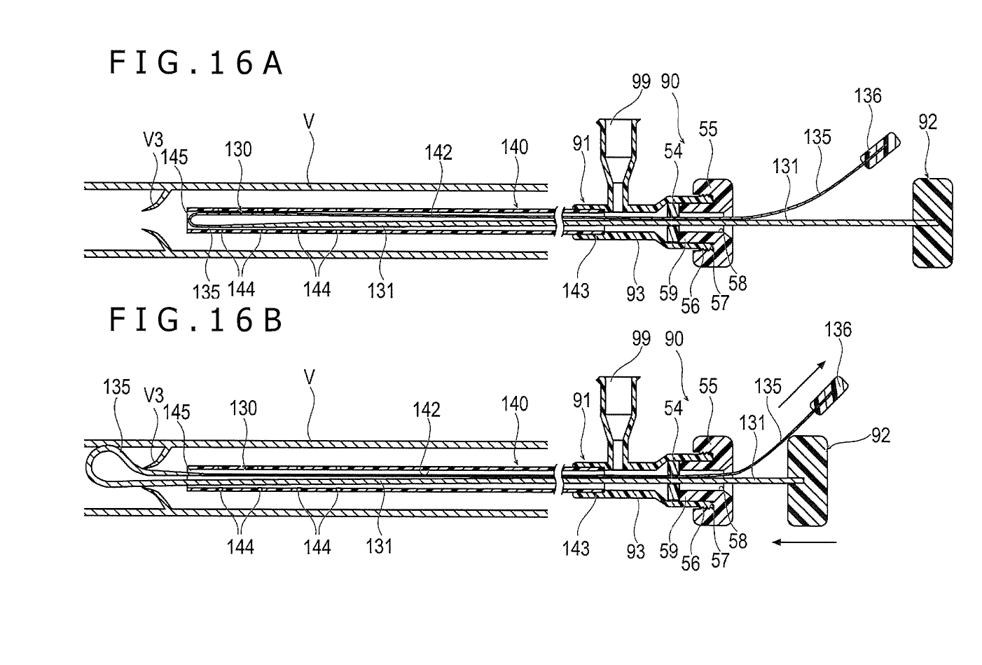

As illustrated by a second modification of the third embodiment in FIGS. 16A and 16B, the contact portion 135 may be a linear material extending while being reduced in diameter from a distal portion of the inside shaft portion 131. Note that parts having functions equivalent or similar to those in the third embodiment are denoted by the same reference signs as used above, and description of those parts is omitted. The contact portion 135 is folded back on the distal side of the inside shaft portion 131 to extend proximally, and extends through the second lumen 142 of the outer sheath 140 and through the first operating portion 91, to be led to the proximal side. An end portion of the contact portion 135 penetrates the first operating portion 91 on the proximal side, and a grip portion 136 which can be grasped by an operator is fixed to the end portion. The contact portion 135 can be accommodated in the outer sheath 140 while being elastically deformed (i.e., compressed radially inwards), as depicted in FIG. 16A. When the second operating portion 92 is moved distally relative to the first operating portion 91 to protrude the contact portion 135 distally from the outer sheath 140 and the grip portion 136 is then moved proximally, the contact portion 135 is bent within the vein V, and is expanded to the radially outer side of the shaft portion 130. Then, the bent contact portion 135 is engaged onto the venous valve V3 or with the branch portion V4. When the operating portion 90 is then rotated, the vein V twists.

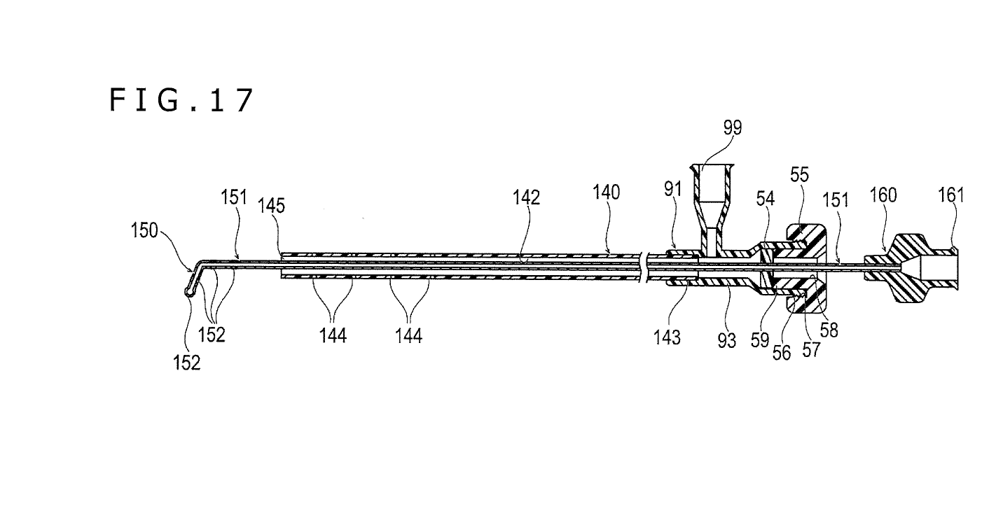

As illustrated by a third modification of the third embodiment in FIG. 17, a contact portion 150 and an inside shaft portion 151 may be formed as a continuous pipe body. An opening or openings 152 for releasing a treatment agent therethrough may be formed in at least part of the contact portion 150 and the inside shaft portion 151. A proximal portion of the inside shaft portion 151 is connected to a second operating portion 160. The second operating portion has an injection port 161 permitting a treatment agent to be injected therethrough. Such a configuration ensures that a syringe or the like filled with the treatment agent may be connected to the injection port 161, and the treatment agent can be released from the contact portion 150 or the inside shaft portion 151, which protrudes from the outer sheath 140.

Note that the present disclosure is not to be limited to the aforementioned embodiments, and various modifications can be made by one skilled in the art, within the scope of this disclosure. For example, in the medical device 1 according to the first embodiment, the treatment agent is released on the distal side of the contact portion 30, while in the medical devices 80 and 120 according to the second and third embodiments, the treatment agent is released on the proximal side of the contact portion 30 or 132. However, the treatment agent may instead be released from a position directly below the contact portion. In addition, the treatment agent may be released from two or more different positions selected from a position on the distal side of the contact portion, a position on the proximal side of the contact portion and a position directly below the contact portion. The step of damaging the inner wall of the vein V by moving the contact portion 30 of the medical device 1 and the step of releasing the treatment agent may be carried out in the reverse order of that described above, or these steps can occur simultaneously.

Further, the configuration of the contact portion is not restricted, so long as the contact portion can change in size, for example, by expanding in the manner of projecting to the radially outer side of the shaft portion.

In the medical devices 1 and 80 according to the first and second embodiments described above, the vein V is damaged by pulling the contact portion 30 proximally. However, the vein V may instead be damaged by pushing the contact portion 30 distally.

As a configuration for expanding the contact portion, the operating portion may be provided with a knock mechanism such that the contact portion is expanded and contracted alternately and repeatedly each time the operating portion is pushed in, like the mechanism provided in a ballpoint pen, for example.

It should be understood by those skilled in the art that various modifications, combinations, sub-combinations and alterations may occur depending on design requirements and other factors insofar as they are within the scope of the appended claims or the equivalents thereof.

The detailed description above describes a blood vessel treatment method. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A blood vessel treatment method comprising:
inserting a medical device into a blood vessel which possesses a lumen, the medical device including an elongate shaft portion and a contact portion configured to contact biological tissue in the blood vessel;
bringing the contact portion into contact with the biological tissue in the blood vessel;
twisting the blood vessel by moving the contact portion; and
releasing a treatment agent from the medical device for occluding or contracting the lumen of the blood vessel.

2. The blood vessel treatment method according to claim 1, wherein during the bringing of the contact portion into contact with the biological tissue in the blood vessel, the contact portion is expanded radially outwardly of the shaft portion so that the contact portion contacts the biological tissue.

3. The blood vessel treatment method according to claim 1, further comprising axially moving the contact portion along the blood vessel while the contact portion is in contact with the biological tissue so as to damage the biological tissue with the contact portion.

4. The blood vessel treatment method according to claim 1,
wherein during the bringing of the contact portion into contact with the biological tissue in the blood vessel, a spiral linear material portion of the contact portion is brought into contact with the biological tissue of the blood vessel, and
during the twisting of the blood vessel by moving the contact portion, the contact portion is moved in an axial direction of the shaft portion to impart a rotating force to the blood vessel by the contact portion, thereby twisting the blood vessel.

5. The blood vessel treatment method according to claim 1, wherein during the twisting of the blood vessel by moving the contact portion, the contact portion is engaged with a branch portion or a valve body of the blood vessel.

6. The blood vessel treatment method according to claim 1, wherein the blood vessel possesses an inner diameter, the inner diameter of the blood vessel being smaller when the blood vessel is twisted by the contact portion than the inner diameter of the blood vessel when the blood vessel is not twisted.

7. The blood vessel treatment method according to claim 6, wherein during the twisting of the blood vessel by moving the contact portion, an inner wall surface of the blood vessel twisted and reduced in inside diameter is brought into contact with the shaft portion.

8. The blood vessel treatment method according to claim 7, wherein during the twisting of the blood vessel by moving the contact portion, the inner wall surface of the blood vessel twisted and reduced in inside diameter is brought into contact with a part of the shaft portion, the part formed with an opening for releasing the treatment agent.

9. The blood vessel treatment method according to claim 1, wherein during the twisting of the blood vessel by moving the contact portion, portions of the inner wall surface of the twisted blood vessel are brought into contact with each other in an overlapping manner.

10. A method comprising:
inserting a medical device into a blood vessel of a living body, the blood vessel possessing an inner diameter;
twisting the blood vessel to reduce the inner diameter of a portion of the blood vessel at which the medical device is located; and
releasing a treatment agent from the medical device while the blood vessel is twisted and the inner diameter of the portion of the blood vessel at which the medical device is located is reduced.

11. The method according to claim 10, wherein the medical device comprises a contact portion and the blood vessel possesses an inner wall, and
the twisting of the blood vessel comprising moving the contact portion of the medical device into contact with the inner wall of the blood vessel and rotating the contact portion to twist the blood vessel so that the inner diameter of the blood vessel is reduced.

12. The method according to claim 11, further comprising moving the contact portion in an axial direction within the blood vessel, while the contact portion is in contact with the inner wall of the blood vessel, to damage the inner wall of the blood vessel.

13. The blood vessel treatment method according to claim 11, wherein
the medical device includes a shaft portion, and
when the blood vessel is twisted, an inner wall surface of the blood vessel is brought into contact with the shaft portion of the medical device.

14. The blood vessel treatment method according to claim 13, wherein the shaft portion of the medical device includes an opening, and the treatment agent is released from the opening of the shaft portion of the medical device.

15. A method comprising:
inserting a contact portion into a living body;
bringing the contact portion into contact with a part of a blood vessel in the living body, the blood vessel possessing a lumen extending throughout its length, the lumen possessing an inner diameter;
rotating the contact portion while the contact portion is in contact with the part of the blood vessel to rotate the part of the blood vessel and cause twisting of the blood vessel, the twisting of the blood vessel reducing the inner diameter of a portion of the blood vessel; and
releasing a treatment agent inside the portion of the blood vessel of reduced inner diameter.
